(19) United States
(12) Patent Application Publication
(10) Pub. No.: US 2018/0371158 A1
(43) Pub. Date: Dec. 27, 2018

(54) PEPTIDOMIMETIC POLYMERS AS CONTROLLED RELEASE MATRICES FOR SMALL MOLECULES, BIOLOGICALS, SYNTHETIC OR SEMI-SYNTHETIC MACROMOLECULES

(71) Applicants: Abraham Joy, Copley, OH (US); Tanmay Jain, Akron, OH (US)

(72) Inventors: Abraham Joy, Copley, OH (US); Tanmay Jain, Akron, OH (US)

(21) Appl. No.: 16/063,470

(22) PCT Filed: Dec. 19, 2016
(86) PCT No.: PCT/US2016/067519

§ 371 Date: Jun. 18, 2018

Related U.S. Application Data
(60) Provisional application No. 62/269,148, filed on Dec. 18, 2015.

(51) Int. Cl. C08G 63/685; A61L 27/18; A61L 27/54; C08G 18/60

(52) U.S. Cl. CPC C08G 63/685; A61L 27/18; A61L 27/54; C08L 67/00; A61L 2300/602; A61L 2300/43; A61L 2430/02; C08G 18/603

(57) ABSTRACT

A controlled release polymer is provided which includes a polymer that has backbone selected from polyesters and polyurethanes, and an amide group with a pendant functional group, where the nitrogen atom of the amide group is part of the polymer backbone, and an active compound dispersed in the polymer. The active compound may be released over time from the controlled release polymer

### CROSS-REFERENCE TO RELATED APPLICATIONS

This application claims priority from U.S. provisional patent application Ser. No. 62/269,148 filed on Dec. 18, 2015 which is incorporated herein by reference in its entirety.

### STATEMENT REGARDING FEDERALLY SPONSORED RESEARCH OR DEVELOPMENT

This invention was made with government support under award number is DMR-1352485 awarded by National Science Foundation. The government has certain rights in the invention.

### FIELD OF THE INVENTION

One or more embodiments provide peptidomimetic urethanes and polyesters used as a matrix for the controlled release of active ingredients.

### BACKGROUND OF THE INVENTION

Various controlled release mechanisms and applications exist. Compared to conventional dosage forms, drug delivery systems based on polymeric material have many advantages, such as reduced toxicity, improved therapeutic effect, convenience, and so on. However, there are still some problems of this strategy, for example, burst release of drugs at the beginning and low efficiency to prepare nano- or microparticles for local or in situ chemotherapy. Methods that can constantly release therapeutic drug amounts over a period of multiple months are preferred. It was found that drugs can be encapsulated into electrospun nanofibers and those systems showed nearly zero-order kinetics of drug release. Devices based on electrospun fibers are promising for drug delivery applications. Other controlled release mechanism that allow for low glass transition temperature (Tg) may also be useful because they allow the encapsulation of the active agent in mild conditions.

Presently there is a need in the art for controlled release polymers that may be used in robust settings.

### SUMMARY OF THE INVENTION

In a first embodiment, the embodiment provides a controlled release matrix comprising a polymer that has backbone selected from polyesters and polyurethanes, and an amide group with a pendant functional group, where the nitrogen atom of the amide group is part of the polymer backbone, and an active compound dispersed in the polymer.

In a second embodiment, the embodiment provides a controlled release matrix as in the first embodiment, wherein the weight percent of the active compound of the total weight of the active compound and polymer is from about 0.1% to about 50%.

In a third embodiment, the embodiment provides a controlled release matrix as in either the first or second embodiment, wherein the functionalized amide polymer includes a unit defined by the formula:

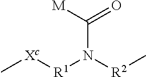

where each X^{c} is an ester group; R¹ and R² may be the same or different and are each hydrocarbon groups; and M is a pendant functional group.

In a fourth embodiment, the embodiment provides a controlled release matrix as in any of the first through third embodiments, wherein the functionalized amide polymer includes a unit defined by the formula:

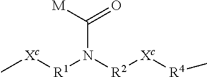

where each X^{c} is a urethane group or an ester group; R¹ and R² may be the same or different and are each hydrocarbon groups; R⁴ is a hydrocarbon group, and M is a pendant functional group.

In a fifth embodiment, the embodiment provides a controlled release matrix as in any of the first through fourth embodiments, wherein functionalized amide polymer is defined by the formula:

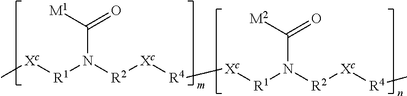

where every X^{c} is a urethane group or every X^{c} is an ester group; R¹ and R² are the same or different and are each hydrocarbon groups; R⁴ is a hydrocarbon group; m and n represent repeating units of the polymer in random or block configuration, and M¹ and M² are different pendant functional groups.

In a sixth embodiment, the embodiment provides a controlled release matrix as in any of the first through fifth embodiments, wherein functionalized amide polymer is defined by the formula:

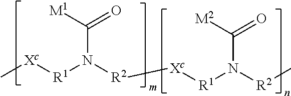

where every X^{c} is a urethane group or every X^{c} is an ester group; R¹ and R² are the same or different and are each hydrocarbon groups; m and n represent repeating units of the polymer in random or block configuration, and M¹ and M² are different pendant functional groups.

In a seventh embodiment, the embodiment provides a controlled release matrix as in any of the first through sixth embodiments, wherein active agent is selected from small molecules, oligomers, and macromolecules.

In an eighth embodiment, the embodiment provides a controlled release matrix as in any of the first through seventh embodiments, wherein active agent is selected from active pharmaceutical ingredients, fluorescent dyes, and imaging agents.

In a ninth embodiment, the embodiment provides a controlled release matrix as in any of the first through eighth embodiments, wherein active agent is selected from peptides, oligonuecleotides, and oligosaccharides.

In a tenth embodiment, the embodiment provides a controlled release matrix as in any of the first through ninth embodiments, wherein where active agent is selected from polysaccharides, proteins, and synthetic polymers.

In an eleventh embodiment, the embodiment provides a controlled release matrix as in any of the first through tenth embodiments, wherein functionalized amide polymer is electrospun.

In a twelfth embodiment, the embodiment method of administering an active agent comprising: supplying a functionalized amide polymer with an active compound dispersed therein to a site in need of the active agent, where the functionalized amide polymer has backbone selected from polyesters and polyurethanes, and an amide group with a pendant functional group, and the nitrogen atom of the amide group is part of the polymer backbone.

In a thirteenth embodiment, the embodiment provides a method of administering an active agent as in any of the first through twelfth embodiments, wherein the weight percent of the active compound of the total weight of the active compound and polymer is from about 0.1% to about 50%.

In a fourteenth embodiment, the embodiment provides a method of administering an active agent as in any of the first through thirteenth embodiments, wherein where the functionalized amide polymer includes a unit defined by the formula:

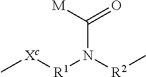

where each X^{c} is an ester group; R¹ and R² may be the same or different and are each hydrocarbon groups; and M is a pendant functional group.

In a fifteenth embodiment, the present invention provides a method of administering an active agent as in any of the first through fourteenth embodiments, wherein where the functionalized amide polymer includes a unit defined by the formula:

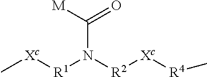

where each X^{c} is a urethane group or an ester group; R¹ and R² may be the same or different and are each hydrocarbon groups; R⁴ is a hydrocarbon group, and M is a pendant functional group.

In a sixteenth embodiment, the present invention provides a method of administering an active agent as in any of the first through fifteenth embodiments, wherein the site in need of the active agent is a bone.

In a seventeenth embodiment, the present invention provides a method of administering an active agent as in any of the first through sixteenth embodiments, where the functionalized amide polymer with an active compound dispersed therein is a medical implant.

In a eighteenth embodiment, the present invention provides a method of administering an active agent as in any of the first through seventeenth embodiments, where the functionalized amide polymer is in the form of a nanoparticles, microparticles, micelles, or vesicle.

In a nineteenth embodiment, the present invention provides a method of administering an active agent as in any of the first through eighteenth embodiments, where the glass transition temperature of the functionalized amide polymer is greater than 37° C.

In a twentieth embodiment, the present invention provides a method of administering an active agent as in any of the first through eighteenth embodiments, where the glass transition temperature of the functionalized amide polymer is less than 22° C.

In a twenty-first embodiment, the present invention provides a method of administering an active agent as in any of the first through eighteenth embodiments, where the glass transition temperature of the functionalized amide polymer is from about 22° C. to about 37° C.

### BRIEF DESCRIPTION OF THE DRAWINGS

FIG. 1 provides a UV-Vis standard curve of rhodamine B in PBS.

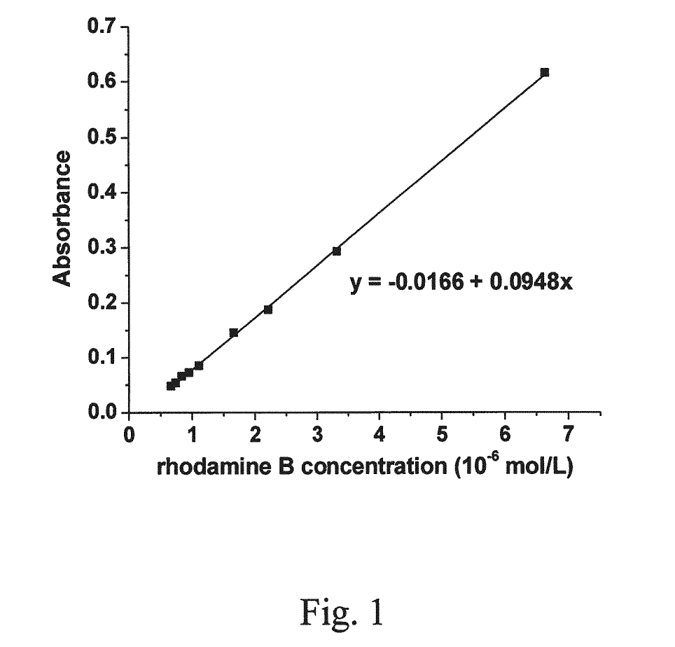

FIG. 2 provides a UV-Vis standard curve of 7-(diethylamino)coumarin-3-carboxylic acid in PBS.

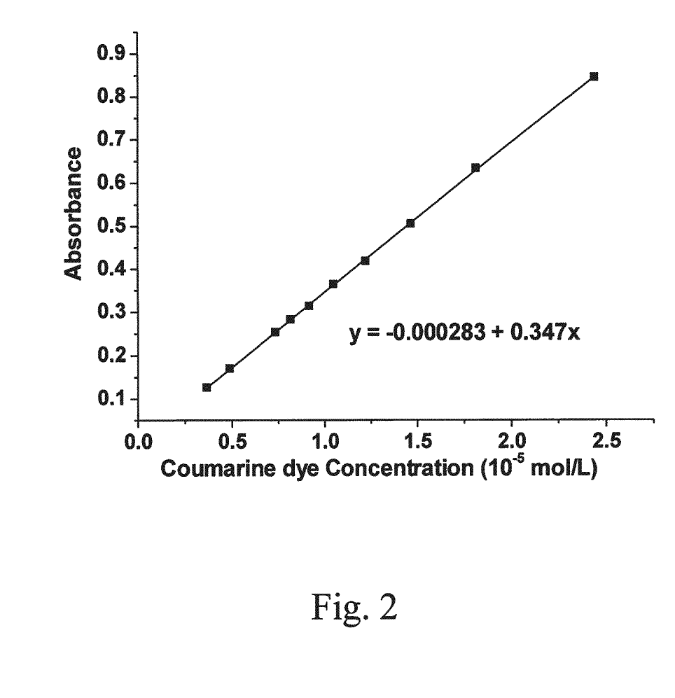

FIG. 3A provides an SEM image of p(mPhe-Keto-BocGlu)/1% Coumarin dye/0.5% Rhodamine B electrospun mat.

FIG. 3B provides a diameter distribution of image of FIG. 3A.

FIG. 3C provides an SEM image of p(mPhe-Keto-BocGlu)-RB/1% Coumarin dye electrospum mat.

FIG. 3D provides a diameter distribution of image of FIG. 3C.

FIG. 4 provides a cumulative release plot of coumarin dye and rhodamine B from fiber mat of p(mPhe-Keto-BocGlu) mixed with rhodamine B and 7-(diethylamino)coumarin-3-carboxylic acid.

FIG. 5 provides a cumulative release plot of coumarin dye and rhodamine B from fiber mat of p(mPhe-Keto-BocGLU)-RB mixed with 7-(diethylamino)coumarin-3-carboxylic acid.

FIG. 6 provides a plot of molecular weights vs days of immersing in 1×PBS.

FIG. 7 provides a cumulative release plot of Dexamethasone from a controlled release matrix of one or more embodiments.

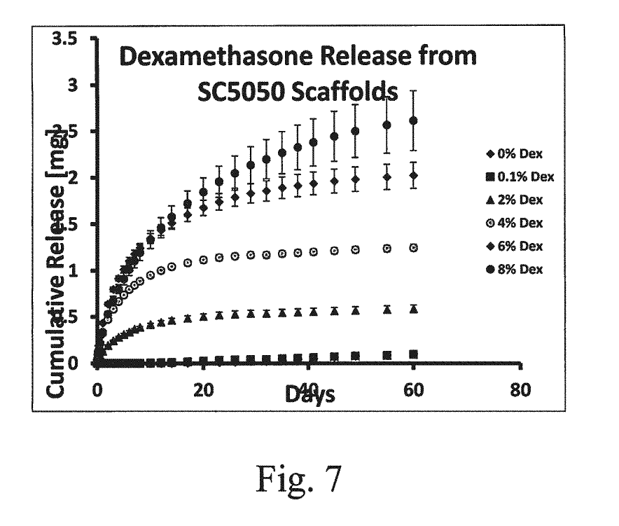

FIG. 8 provides structure for the polymers SC5050.

### DETAILED DESCRIPTION OF ILLUSTRATIVE EMBODIMENTS

In one or more embodiments, a peptidomimetic polymer is provided, which may also be referred to as a functionalized amide polymer. A peptidomimetic polymer may comprise, a polymer backbone selected from polyesters and polyurethanes; and an amide group with a pendant functional group, where the nitrogen atom of the amide group is part of the polymer backbone. Advantageously, it has been found that functionalized amide polymers may be use as a controlled release matrix for active ingredients such as small molecules, biologicals, and synthetic or semi-synthetic macromolecules.

As previously mentioned, a functionalized amide polymer includes a nitrogen atom of the amide group that is part of the polymer backbone, such that polymers herein can be generally conceptualized by the structure below:

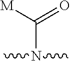

where [symbol] generally represents a polymer backbone selected from polyesters and polyurethanes; and M is a functional group. Suitable functionalized amide polymers and there preparation are described in U.S. Pat. Publ. No. 2015/0094422, U.S. Pat. Publ. No. 2016/0024251, and WO2016049029, all of which is incorporated herein by reference.

In one or more embodiments, the pendant functional groups of the functionalized amide polymers and end-functionalized amide compound having a pendant functional group, which are described below may be an organic group. In some embodiments, the pendant functional group is a group capable of reacting with other reagents to provide a desired functionality in a post-polymerization functionalization step that will be described herein. In other embodiments, the pendant functional group includes a protecting group that protects the M group from reacting with other reagents during monomer creation or polymer creation or both or during post functionalization steps, particularly with multifunctional polymers as described herein.

Representative examples of organic pendant functional groups include but are not limited to:

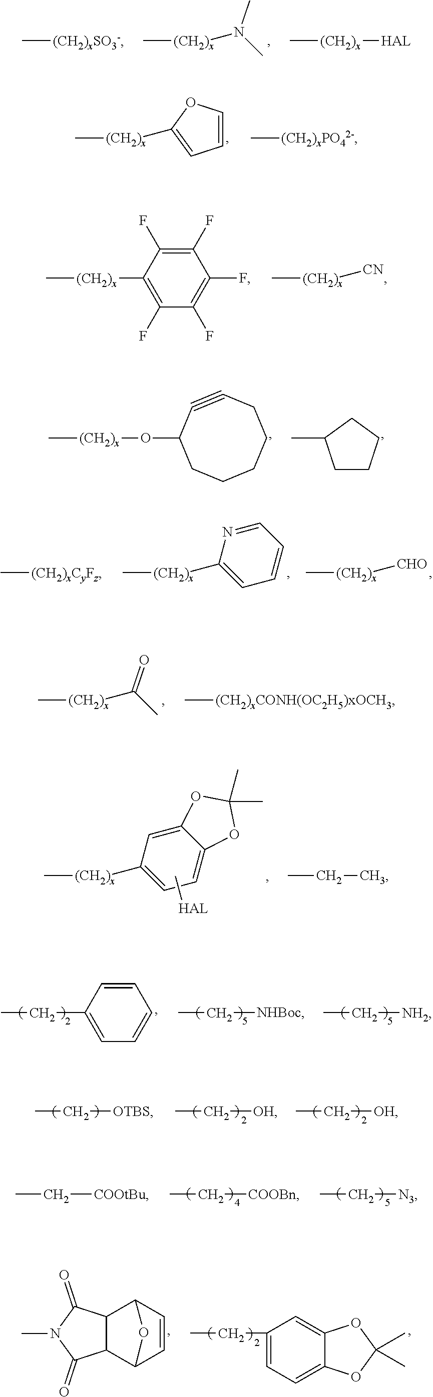

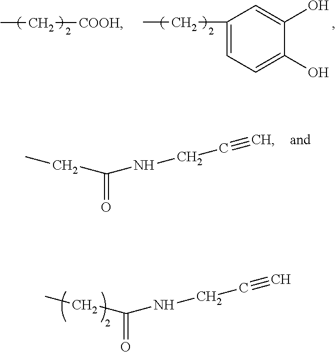

wherein x is from 0 to 6, and HAL denotes a halogen. It is specifically noted that, thought specific number are provided for repeating unit of —CH2— in some of the above structures, the present invention can be practice with x repeating units of those structures. Herein, it will be understood that Boc stands for tert-butyloxycarbonyl, TBS stands for ditertbutyl dimethylsilyl, tBu stands for t-Butyl, Bn stands for benzyl.

Representative examples of groups suitable for post polymerization functionalization include groups with azide, carboxylic acid, hydroxyl, amine, nitrile, furan, aldehyde/ketone, maleimide, propargyl, or halogen functionality. Particular non-limiting examples include:

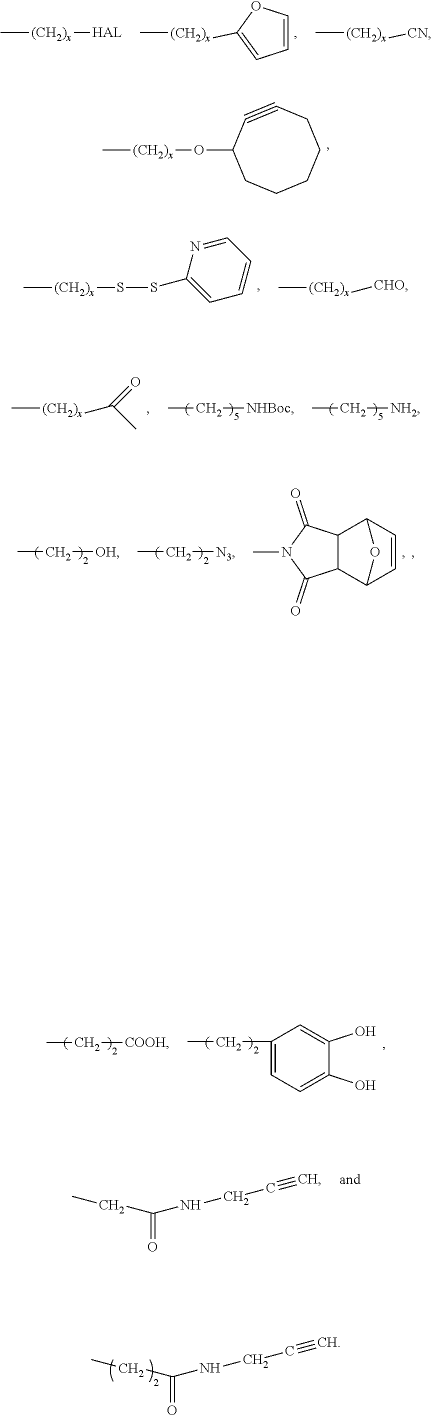

Representative examples of protected groups include M groups protected with tert-butyloxycarbonyl, pyridyl disulfide, t-Butyl, benzyl, ketal, and ditertbutyl dimethylsilyl. Non-limiting examples include:

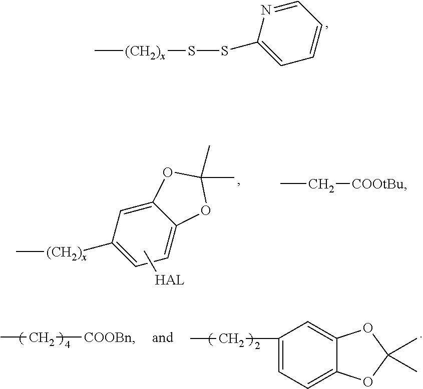

In one or more embodiments, M may be an amino acid side chain. An amino acid side chain is a group that includes a terminal functional group of an amino acid. In one or more embodiments, the terminal functional group of the amino acid is attached to a carbon chain or connecting group. In some embodiments, the carbon chain is a different length than the chain of the corresponding amino acid. Representative examples of residues of an amino acid side chain include, but are not limited to:

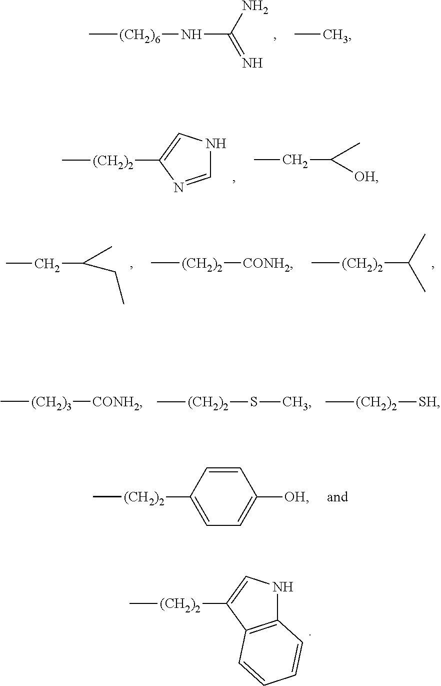

In one or more embodiments, a functionalized amide polymer may be prepared by polymerizing an end-functionalized amide compound having a pendant functional group. In one or more embodiments, the end functionalized amide compound having a pendant functional group may be defined by the formula:

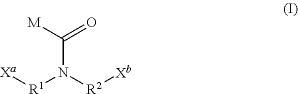

where X^{a} and X^{b} may be the same or different and are each selected from a hydroxyl group and a carboxylic acid group; R¹ and R² may be the same or different and are each hydrocarbon groups; and M is a pendant functional group. In one or more embodiments, where one of X^{a} and X^{b} is a hydroxyl group while the other of X^{a} and X^{b} is a carboxylic acid group, the end-functionalized amide compound may be referred to as a hydroxy acid amide compound having a pendant functional group. In one or more embodiments, where both of X^{a} and X^{b} are hydroxyl groups end-functionalized amide compound may be referred to as a diol amide compound having a pendant functional group. In one or more embodiments, where both of X^{a} and X^{b} are carboxylic acid groups, the end-functionalized amide compound may be referred to as a dicarboxylic acid amide compound having a pendant functional group.

In one or more embodiments, suitable hydrocarbon groups for use in the R¹ and R² of the end-functionalized amide compound having a pendant functional group and resultant polymers may be linear, cyclic, or branched hydrocarbon groups. In one or more embodiments, the hydrocarbon groups may include 1 to 10 carbon atoms, in other embodiments 2 to 8 carbon atoms, and in other embodiments, 3 to 6 carbon atoms.

In one or more embodiments, the functionalized amide polymer may be prepared from an amide functional diol compounds and optionally at least one comonomer. Suitable co-monomers include diisocyanates, dicarboxylic acids, hydroxy acids, and diols.

Suitable dicarboxylic acids useful as copolymers may be defined by the formula:

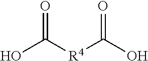

where R⁴ is an organic group.

Advantageously, the organic group R⁴ may be selected to tailor the properties of the thermoresponsive polyester. In one or more embodiments, the hydrophobicity of the polymer may be adjusted by varying the organic group R⁴. For example, the hydrophobicity may be adjusted by altering the chain length of R⁴ or adding a functional group. In one or more embodiments, R⁴ may be a hydrocarbon group with 1 to 10 carbon atoms. In other embodiments, R⁴ may be a polyoxyethylene (O—CH₂—CH₂)ₙ group. Suitable molecular weights of polyoxyethylene groups may be from 200 to 4000, in other embodiments 200 to 2000, and in still other embodiments 200 to 1000. In other embodiments, R⁴ may include a functional group. In these embodiments, the dicarboxylic acids may be referred to as a functional dicarboxylic acids. In certain embodiments, were R⁴ includes a functional group the functional group may be pendantly attached to the dicarboxylic acid. Functional groups may be added to alter the hydrophobicity or add a selectivity to the resultant thermoresponsive polyester. Specific examples of dicarboxylic acids with functional groups include glutamic acid, malic acid, tartaric acid etc., where the amine or hydroxyl group is protected prior to polymerization. Those skilled in the art will appreciate that these functional groups of the functional dicarboxylic acids may be protected. Suitable protecting groups include, but are not limited to Tetrahydropyranyl (THP), tert-butyldimethylsilyl (TBDMS), trimethylsilyl (TMS), and tert-Butoxy carbamate (Boc).

Suitable diisocyanates useful as copolymers may be defined by the formula:

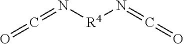

where R⁴ is an organic group.

Similarly with the dicarboxylic acids, the organic group R⁴ of the diisocyanate may be selected to tailor the properties of the thermoresponsive polyester such as hydrophocity and selectivity. In one or more embodiments, R⁴ may be a hydrocarbon group with 1 to 10 carbon atoms. In other embodiments, R⁴ may be a polyoxyethylene (O—CH₂—CH₂)ₙ group. Suitable molecular weights of polyoxyethylene groups may be from 200 to 4000, in other embodiments 200 to 2000, and in still other embodiments 200 to 1000. In other embodiments, R⁴ may include a functional group. In these embodiments, the diisocyanate may be referred to as a functional diol. In certain embodiments, were R⁴ includes a functional group the functional group may be pendantly attached to the diisocyanate.

Suitable diols useful as copolymers may be defined by the formula:

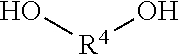

where R⁴ is an organic group.

Similarly with the dicarboxylic acids, the organic group R⁴ of the diol may be selected to tailor the properties of the thermoresponsive polyester such as hydrophocity and selectivity. In one or more embodiments, R⁴ may be a hydrocarbon group with 1 to 10 carbon atoms. In other embodiments, R⁴ may be a polyoxyethylene (O—CH₂—CH₂)ₙ group. Suitable molecular weights of polyoxyethylene groups may be from 200 to 4000, in other embodiments 200 to 2000, and in still other embodiments 200 to 1000. In other embodiments, R⁴ may include a functional group. In these embodiments, the diol may be referred to as a functional diol. In certain embodiments, were R⁴ includes a functional group the functional group may be pendantly attached to the diol.

In certain embodiments, the diol may include an ester group. In these or other embodiments, the inclusion of ester groups via inclusion of comonomers of diols with ester groups may be useful to improve or provide polymer degradation for certain embodiments where the functionalized amide polymer is used in a controlled release matrix. For example, a when the functionalized amide polymer is a polyurethane, inclusion of diols during the polymerization that include an ester group will provide ester locations in the final polymer where degradation may occur. Examples of suitable diols that include ester groups may be found in U.S. Pat. Publ. No. US20160002389, which is incorporated herein by reference.

In one or more embodiments, a diol that includes an ester group may be defined by the formula

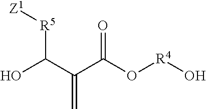

where R⁴ is a hydrocarbon group; R⁵ is a bond or a hydrocarbon group; and Z¹ is hydrogen, protected amine, protected carboxylic acid, protected hydroxyl, alkoxy, or silyloxy group. Another diol that includes an ester group may be defined by the formula

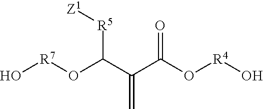

where R⁴ is a hydrocarbon groups; R⁵ is a bond or a hydrocarbon group; R⁷ hydrocarbon group; and Z¹ is hydrogen, protected amine, protected carboxylic acid, protected hydroxyl, alkoxy, or silyloxy group.

In certain embodiments, the comonomer may include a crosslinkable group. In one or more embodiments, the crosslinkable group may be a photoresponsive. Exemplary monomers that include photoresponsive crosslinkable groups include photoactive coumarin monomers, examples of which may be found in WO2014074845, encorporated herein by reference. In one or more embodiments, a comonomer with a crosslinkable coumarin group may be defined by the formula

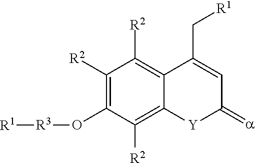

where each R¹ is individually an alcohol, a carboxylic acid, or an isocyanate, each R² is individually a hydrogen atom, a bromine atom, an iodine atom, or a methoxy group; R³ is a hydrocarbon group; Y is an oxygen atom or a nitrogen atom with an organic substitution; and a is an oxygen atom or a sulfur atom.

In one or more embodiments, where the functionalized amide polymer is prepared with a hydroxy acid amide compound having a pendant functional group, the functionalized amide polymer may include a unit defined by the formula:

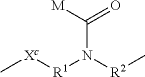

where each X^{c} is an ester group; R¹ and R² may be the same or different and are each hydrocarbon groups; and M is a pendant functional group.

In one or more embodiments, where the functionalized amide polymer is prepared with a dicarboxylic acid amide compound having a pendant functional group or a diol amide compound having a pendant functional group, the functionalized amide polymer may include a unit defined by formula defined by the formula:

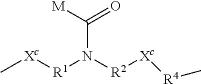

where each X^{c} is a urethane group or an ester group; R¹ and R² may be the same or different and are each hydrocarbon groups; R⁴ is a hydrocarbon group, and M is a pendant functional group.

The functionalized amide polymer may include multiple functionalized amide units. In one or more embodiments, the functionalized amide polymer may include two or more different amide units. In these or other embodiments, the two or more different amide units may be in random or block configurations. In one or more embodiments, the two or more pendant functional groups may be a result of polymerizing two or more end-functionalized amide compounds having different pendant functional groups. In other embodiments, the two or more pendant functional groups may be a result post-polymerization modification of the pendant functional groups

In one or more embodiments, where the functionalized amide polymer is prepared using end-functionalized amide compounds having a pendant functional group and a co-monomer the functionalized amide polymer may be defined by the formula:

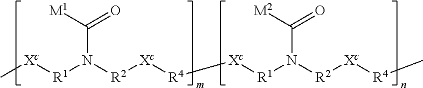

where every X^{c} is a urethane group or every X^{c} is an ester group; R¹ and R² may be the same or different and are each hydrocarbon groups; R⁴ is a hydrocarbon group; m and n represent repeating units of the polymer in random or block configuration, and M¹ and M² are different pendant functional groups.

In particular embodiments, where the functionalized amide polymer is prepared using diol functionalized amide compounds having a pendant functional group and a dicarboxylic acid the functional amide polymer may be a multifunctional polyester defined as below:

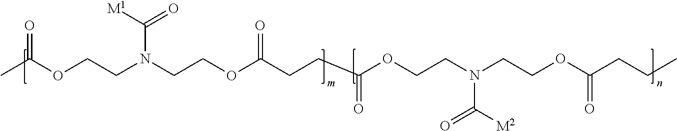

wherein m and n represent repeating units of the polymer in random or block configuration and M¹ and M² are different pendant functional groups.

In certain embodiments, where the functionalized amide polymer is prepared with diol functionalized amide compound having a pendant functional group and a diisocyanate the functional amide polymer may be a multifunctional polyester defined as below:

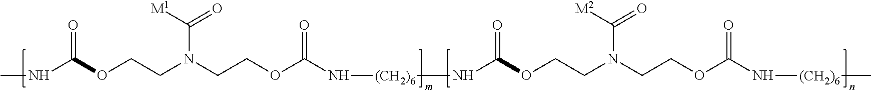

wherein m and n represent repeating units of the polymer in random or block configuration and M¹ and M² are different pendant functional groups.

In one or more embodiments, where the functionalized amide polymer is prepared using a hydroxy acid amide compounds having a pendant functional group, the functionalized amide polymer may be defined by the formula:

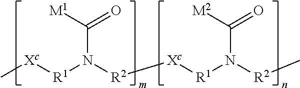

where every X^{c} is a urethane group or every X^{c} is an ester group; R¹ and R² may be the same or different and are each hydrocarbon groups; m and n represent repeating units of the polymer in random or block configuration, and M¹ and M² are different pendant functional groups.

In certain embodiments, where the functionalized amide polymer is prepared using a hydroxy acid amide compounds having a pendant functional group, the functionalized amide polymer may be defined as below:

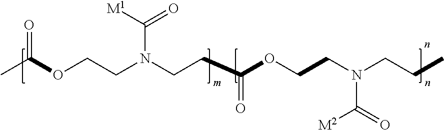

wherein m and n represent repeating units of the polymer in random or block configuration and M¹ and M² are different pendant functional groups.

While depicted above as a polymer with one or two different amide units, the functionalized amide polymer may in other embodiments have more than two amide units. In one or more embodiments, the functionalized amide polymer may have 3, 4, 5, 6, 7, 8, 9, 10, or more different amide units.

The molecular weight of the functionalized amide polymer may be determined through size exclusion chromatography. In one or more embodiments, the functionalized amide polymers are made to have a number average molecular weight from about 10,000 g/mol to about 200,000 g/mol, in other embodiments, from about 20,000 g/mol to about 100,000 g/mol, and in other embodiments from about 30,000 g/mol to about 80,000 g/mol. In one or more embodiments, the polydispersity of the functionalized amide polymer polyesters may range from about 1.1 to about 2.5.

Those skilled in the art will appreciate that the glass transition temperature (Tg) of the functionalized amide polymer may be controlled or adjusted through the selection of constituents when preparing the polymer. For example, the size and type of pendant functional group or intermolecular forces between the pendant functional groups may be used to vary the glass transition temperature of the functionalized amide polymer.

In one or more embodiments, the functionalized amide polymer may be characterized by a glass transition temperature, which may be measured by differential scanning calorimetry. In one or more embodiments, the glass transition temperature may be at most 75° C., in other embodiments at most 70° C., and in other embodiments, at most 65° C. In one or more embodiments, the glass transition temperature may be at least −50° C., in other embodiments at least −30° C., and in other embodiments at least −15° C. In certain embodiments, the functionalized amide polymer may have a viscous, honey-like, consistency, which may be referred for the purpose of this specification as a low glass transition temperature. In several instances, low Tg polymers are advantageous for encapsulation and release of small molecules, biologicals, oligomers, synthetic or semi-synthetic macromolecules. The low glass transition temperature (Tg) allows encapsulation of the active agent in mild conditions such as low temperatures and/or without the use of solvents. These active agent encapsulated low Tg polymers can be extruded into the desired form, can be injected through syringe thereby avoiding the need for surgery, and can be delivered to a local physiological site without having the active agent circulating throughout the body. In these or other embodiments, the glass transition temperature may be from about −50° C. to about 22° C., in other embodiments from about −30° C. to about 10° C., and in other embodiments, at from about −15° C. to about 0° C. In still other embodiments, the glass transition temperature may be from about 22° C. to about 37° C., in other embodiments from about 25° C. to about 35° C., and in other embodiments, at from about 28° C. to about 30° C.

In other embodiments, the functionalized amide polymer may have a solid constancy that may or may not be flexible, which may be referred for the purpose of this specification as a high glass transition temperature. In these or other embodiments, the glass transition temperature may be from about 37° C. to about 75° C., in other embodiments from about 40° C. to about 65° C., and in other embodiments, at from about 45° C. to about 60° C.

In one or more embodiments, the controlled release matrix of functionalized amide polymer may be characterized by the length of time sustained release of an active agent. In one or more embodiments, an active agent may be released for at least 1 day, in other embodiments for at least 3 days, and in other embodiments for at least 10 days. In these or other embodiments, an active agent may be released for at most 3 years, in other embodiments for at most 1 year, and in other embodiments for at most 90 days. In one or more embodiments, an active agent may be released from about 1 day to about 3 years, in other embodiments from about 3 days to about 1 year, and in other embodiments from about 10 days to about 90 days.

Active agents or active compounds are molecules that provides some activity that has a benefit for the environment of which the controlled release matrix is placed. Molecules suitable for use as active agents in a controlled release matrix include small molecules, oligomers, and macromolecules. Suitable small molecules for encapsulation include active pharmaceutical ingredients, fluorescent dyes, and imaging agents. Suitable oligomers for encapsulation include peptides, oligonuecleotides, and oligosaccharides. Suitable macromolecules for encapsulation include polysaccharides, proteins, and synthetic polymers.

In one or more embodiments the controlled release matrix may be defined by weight percent of the active compound. In these or other embodiments, the weight percent of the active compound of the total weight of the active compound may be at least 0.1%, in other embodiments, at least 0.5%, in other embodiments at least 1% in other embodiments at least 2%, in other embodiments at least 3%, in other embodiments at least 4%, in other embodiments at least 5%, in other embodiments at least 10%. In these or other embodiments, the weight percent of the active compound of the total weight of the active compound may be at most 50%, in other embodiments at most 45%, in other embodiments at most 40% in other embodiments at most 35%, in other embodiments at most 30%, in other embodiments at most 25%, in other embodiments at most 20%, in other embodiments at most 15%. In one or more embodiments, the weight percent of the active compound of the total weight of the active compound may be from about 0.1% to about 50%, in other embodiments at from about 0.5% to about 45%, in other embodiments at from about 1% to about 40%, in other embodiments at from about 2% to about 35%, in other embodiments at from about 3% to about 30%, in other embodiments at from about 4% to about 25%, in other embodiments at from about 5% to about 20%, and in other embodiments at from about 10% to about 15%.

As noted above functionalized amide polymers may be used in a controlled release matrix. Advantageously, it has been found that he functionalized amide polymers may release active agents over an extended period of time. In one or more embodiments, the active agents may be released by diffusing through the functionalized amide polymer matrix. In other embodiments, the active agents may be released from the functionalized amide polymer matrix when the functionalized amide polymer matrix degrades. In still other embodiments, the active agents may be released by a combination of diffusing through the functionalized amide polymer matrix ab by degradation of the functionalized amide polymer matrix.

The controlled release matrix and addition of the active agent to the controlled release matrix may be performed by multiple methods.

In one or more embodiments, a controlled release matrix containing an active agent may be prepared by electrospinning. In these or other embodiments, the active agent may be dissolved in a suitable solvent along with a functionalized amide polymer. The controlled release matrix containing an active agent is prepared upon electrospinning the mixture. In these or other embodiments, the controlled release matrix containing an active agent may be in the form of a fiber or a mat of fibers. The use of electrospinning is particularly useful for high glass transition temperature functionalized amide polymers.

In other embodiments, a controlled release matrix containing an active agent may be prepared by dissolving a functionalized amide polymer in an appropriate solvent along with an active agent. Upon removing the solvent, the active agent is dissolved or dispersed in the polymer matrix. In certain embodiments, the solvent may be water or an aqueous solvent and the functionalized amide polymer may be water soluble. The polymer and active agents then be processed by multiple different methods such as solvent casting, compressed molding, jet spinning, pressure spraying, dispersions or phase-separation. The polymer and active agents can be formulated as nanoparticles and/or microparticles, micelles, vesicles, dispersions or aerosols.

In one or more embodiments, particularly where the functionalized amide polymer is a low glass transition temperature functionalized amide polymer, the active agent may be mixed into the functionalized amide polymer. The mixing may be performed with or without the aid of a solvent. In certain embodiments, such as those where the functionalized amide polymer includes a crosslinking agent, the functionalized amide polymer may be extruded. In those embodiments, where the functionalized amide polymer includes a crosslinking agent the crosslinking may be performed aft the extrusion. In more specific embodiments, a functionalized amide polymer includes a crosslinking agent may be extruded using a 3D printer and then crosslinked to maintain its form.

In one or more embodiment, a controlled release matrix of functionalized amide polymers may be used to prepare a wound dressing. The wound dressings may optionally have a backing. In one or more embodiments, the wound dressing backing may provide one or more advantageous features such as rigidity permeable gas exchange, and limited gas exchange with the open atmosphere. In these or other embodiments, an adhesive may be applied to the backing of the wound dressing.

In one or more embodiments, the controlled release matrix of functionalized amide polymers may be used as a medical implant. In one or more embodiment, a controlled release matrix of functionalized amide polymers may be used to promote bone growth. The controlled release matrix, which may optionally include a source of calcium may, include as a active agent therapeutics that may improve bone healing signaling cues to the body or antibacterials to avoid infections.

While particular embodiments of the invention have been disclosed in detail herein, it should be appreciated that the invention is not limited thereto or thereby inasmuch as variations on the invention herein will be readily appreciated by those of ordinary skill in the art. The scope of the invention shall be appreciated from the claims that follow.

### Examples

The experimental section describes the chemicals needed for synthesis of targeted product, the method of making monomers and polymers and the characterization and tests of materials.

### Materials and Instrumentation

Materials: All the reagents were purchased from Sigma Aldrich or Alfa Aesar and used without further purification unless otherwise noted. N,N′-diisopropylcarbodiimide (DIC, 99%) was purchased from Oakwood Chemical and used as received. 4-(dimethylamino) pyridinium 4-toluene sulfonate (DPTS) was prepared according to literature methods. Dichloromethane was dried by distillation over anhydrous CaH₂ and the DMF was dried by distillation over anhydrous CaH₂. Silica gel (40-63 μm, 230×400 mesh) for flash chromatography was purchased from Sorbent Technologies, Inc.

Instrumentation: All ¹H and ¹³C NMR spectra of the monomers and polyesters were recorded on either a Varian Mercury 300 MHz or 500 MHz spectrometer. Chemical shifts were recorded in ppm (δ) relative to solvent signals. Polyester molecular weights were analyzed on a TOSOH EcoSec HLC-8320 GPC equipped with a refractive index detector (RI) and UV detector. Separation occurred over two PSS Gram Analytical GPC Columns in series using 25 mM LiBr in dimethylformamide (DMF) as eluent at a flow rate of 0.8 mL/min. The column and detector temperatures were maintained at 50° C. Molecular weights were obtained relative to PS standards using the RI signal. Thermogravimetric analysis (TGA) was performed with TA Q500 over an interval of ambient temperature to 600° C. at a heating rate of 10° C./min under a N₂ atmosphere. Differential scanning calorimetry (DSC) was performed on TA Q2000 DSC with a liquid N₂ cooling unit and a heating/cooling rate of 10° C./min. ESI MS was performed on Bruker HTC ultra QIT.

### Synthesis of Rhodamine B Derivative

Synthesis of M4.1: tert-butyl-N-hydroxycarbamate (2.0 g, 15 mmol) was added to a 100 ml round bottom flask. 10 mL anhydrous DMF was added to the flask. 1,8-Diazabicyclo [5.4.0]undec-7-ene (DBU) (2.23 mL, 15 mmol) dissolved in 5 mL was added to the above solution. 1,6-dibromohexane (4.61 mL, 30 mmol) dissolved in 5 mL anhydrous DMF was added to the above solution. After 3 h of stirring at RT, an additional 1 mL of 1, 8-diazabicyclo[5.4.0]undec-7-ene (DBU) was added. After an additional 2 h of stirring at RT, the temperature was increased to 50° C. and allowed to stir for 24 h. The reaction mixture was then concentrated by rotary evaporation. The residue was dissolved in 9:1 ethyl acetate:methanol and filtered through a plug of silica gel to remove the DBU HBr salt. The filtrate was collected and the solvent was evaporated by rotary evaporation. The crude oil was purified by silica gel column chromatography with a 0-10% hexane in ethyl acetate gradient eluting system. The product was dried through rotary evaporation and high vacuum and characterized via 1H NMR (500 MHz, CDCl3): δ 7.08 (s, 1H), 3.86 (t, J=6.60 Hz, 2H), 3.42 (t, J=6.85 Hz, 2H), 1.82-1.94 (m, 2H), 1.59-1.71 (m, 2H), 1.50 (s, 9H).

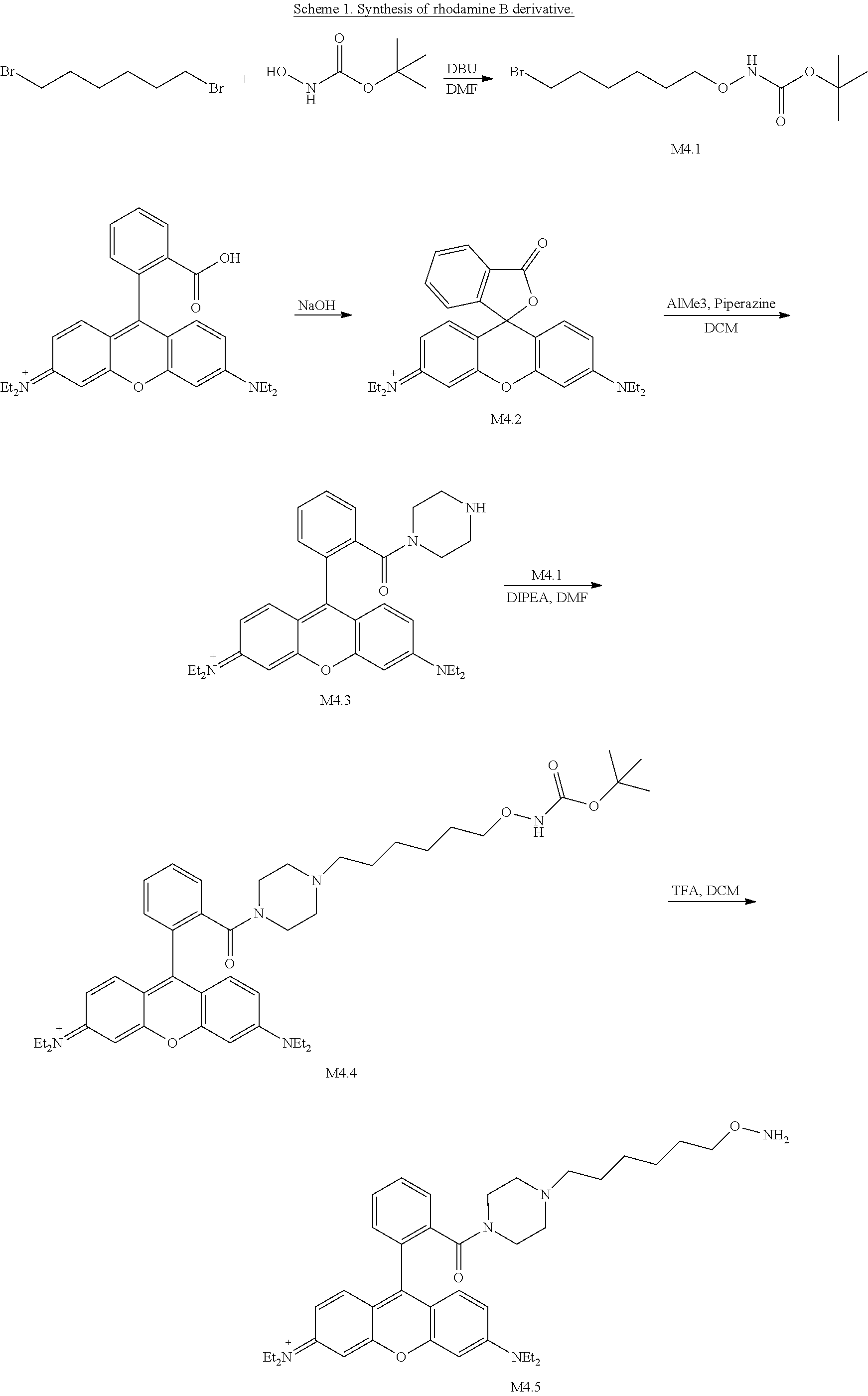

Synthesis of M4.2: Rhodamine B (1.0 g) was dissolved in 100 ml 1 M sodium hydroxide solution and stirred for 2 h. The solution was partitioned with DCM (100 mL). The organic layer was isolated and the aqueous layer was extracted twice with DCM. Combined organic layers were washed once with 1 M sodium hydroxide and brine. The organic solution was dried with anhydrous Na₂SO₄, filtered, concentrated with rotary evaporation and dried under high vacuum to yield 0.8 g of Rhodamine B base as a pink foam. ¹H NMR (300 MHz, CD₃OD): δ 8.04-8.18 (m, 1H), 7.54-7.75 (m, 2H), 7.20-7.40 (m, 3H), 6.99 (dd, J=2.49, 9.51 Hz, 2H), 6.91 (d, J=2.34 Hz, 2H), 3.66 (q, J=7.03 Hz, 8H), 1.30 (t, J=7.03 Hz, 12H).

Synthesis of M4.3: A two neck flask and addition funnel were flame dried before use. Piperazine (2.32 g, 27 mmol) was added into the flask and vacuum backfilled with N₂ three times. 12 mL anhydrous DCM was added. Trimethylaluminium (6.7 mL 2M in toluene, 13.5 mmol) was added to the flask dropwise through an additional funnel and the reaction was stirred for 1 h to yield a white precipitate. A solution of rhodamine B base (2.98 g, 6.7 mmol) in 10 mL of anhydrous DCM was added dropwise to the heterogeneous solution. The mixture was refluxed for 24 h. A 0.1 M aqueous solution of HCl was added dropwise until gas evolution ceased. The heterogeneous solution was filtered and the retained solids were rinsed with DCM and a 4:1 DCM/MeOH solution. The combined filtrates were concentrated. The residue was dissolved in DCM, filtered to remove insoluble salts, and concentrated again. The resulting solid was then partitioned between dilute aqueous NaHCO₃ (150 mL) and EtOAc (100 mL). After isolation, the aqueous layer was washed with three additional portions of EtOAc (100 mL) to remove residual starting material. The retained aqueous layer was saturated with NaCl, acidified with 1 M aqueous HCl, and then extracted with multiple portions of 2:1 isopropanol/dichloromethane until a faint pink color persisted. The combined organic layers were then dried over Na₂SO₄, filtered, and concentrated by rotary evaporation. The purple solid was dissolved in a minimum amount of MeOH and precipitated by dropwise addition to a large volume of diethyl ether. The product was collected by filtration as a dark purple solid (3.0 g, 87%). ¹H NMR (500 MHz, CD₃OD): δ 7.73-7.86 (m, 3H), 7.48-7.58 (m, 1H), 7.27 (d, J=9.54 Hz, 2H), 7.11 (dd, J=2.20, 9.54 Hz, 2H), 6.98 (d, J=2.20 Hz, 2H), 3.58-3.80 (m, 12H), 3.13 (br. s., 4H), 1.97-2.05 (m, 1H), 1.32 (t, J=7.09 Hz, 12H).

Synthesis of M4.4: Compound M4.3 (113 mg, 0.206 mmol) and 500 μL anhydrous DMF was added to a 20 mL scintillation vial with a magnetic stir bar. Compound M4.1 (76.4 mg, 0.258 mmol), N, N-diisopropylethylamine (DIPEA) (62.5 μL, 0.368 mmol), and an additional portion of anhydrous DMF (500 μL) were added to the vial. The reaction mixture was stirred at rt. for 20 h. An additional aliquot of compound M4.1 (76.4 mg, 0.258 mmol) and DIPEA (63 μL, 0.368 mmol) and anhydrous DMF (300 μL) was then added. An additional aliquot of compound M4.1 (76.4 mg), DIPEA (63 μL), and DMF (180 μL) was then added after another 18 h. After an additional 18 h, the DMF was removed under reduced pressure, and the remaining crude mixture was partitioned between ethyl acetate and saturated sodium bicarbonate. The aqueous layer was extracted with a 1:3 mixture of isopropanol and dichloromethane until colorless, and the organic layer was collected, dried over anhydrous sodium sulfate, filtered, and concentrated to afford 68 mg (46%) of dark purple solid. ¹H NMR (500 MHz, CD₃OD): δ 7.70-7.81 (m, 2H), 7.59-7.70 (m, 1H), 7.45-7.56 (m, 1H), 7.23-7.32 (m, 2H), 7.07 (d, J=9.54 Hz, 2H), 6.98 (br. s., 2H), 3.65-3.87 (m, 10H), 3.33-3.50 (m, 8H), 2.15-2.29 (m, 3H), 1.54-1.67 (m, 2H), 1.37-1.48 (m, 11H), 1.23-1.37 (m, 16H).

Synthesis of M4.5: Compound M4.4 (10.5 mg, 0.0144 mmol) was added to a 20 mL scintillation vial. A mixture of 1 to 1 ratio of trifluoroacetic acid (TFA) and dichloromethane (1 mL) was added at room temperature. The mixture was allowed to stand for 2 min. The solvent was then evaporated under a stream of nitrogen gas. The following procedure was repeated three times. The crude residue was dissolved in 1 mL of dichloromethane and concentrated under a stream of nitrogen to yield 7.3 mg (80%) of dark purple solid. MS ESI: M⁺ calculated: 626.4, found: 626.3.

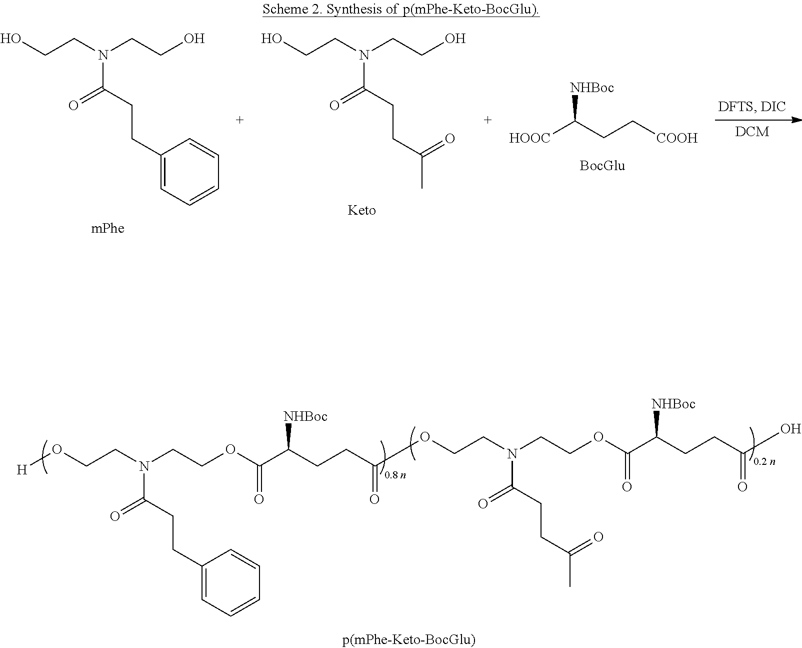

### Synthesis of p(mPhe-Keto-BocGlu)

mPhe (4.2234 g, 17.8 mmol), Keto monomer (0.9043 g, 4.45 mmol), Boc-L-Glutamic acid (5.5006 g, 22.2 mmol), and DPTS (2.6011 g, 8.9 mmol) were added to a Schlenk flask and vacuum backfilled with N₂ three times. 20 mL anhydrous DCM was added to the flask by syringe. The reaction mixture warmed up to 40° C. for 1-2 min, and then cooled to 0° C. DIC (11 mL, 70 mmol) was added dropwise by syringe and reaction mixture was warmed to RT and stirred for 72 h. Then polymer was precipitated twice from methanol and dried under vacuum. 4.2 g (42.9%) polymer was obtained. ¹H NMR (500 MHz, CDCl₃): δ 7.22 (br. s., 4H), 5.30 (br. s., 0.84H), 4.12-4.32 (m, 5H), 3.54-3.61 (m, 4H), 2.97 (br. s., 1.61H), 2.66 (br. s., 2.46H), 2.37 (br. s., 2H), 2.14-2.21 (m, 1.64H), 1.78-2.01 (m, 1H), 1.42 (br. s., 9H). Mₙ=79 kDa, M_{w}=106 kDa, PDI=1.34.

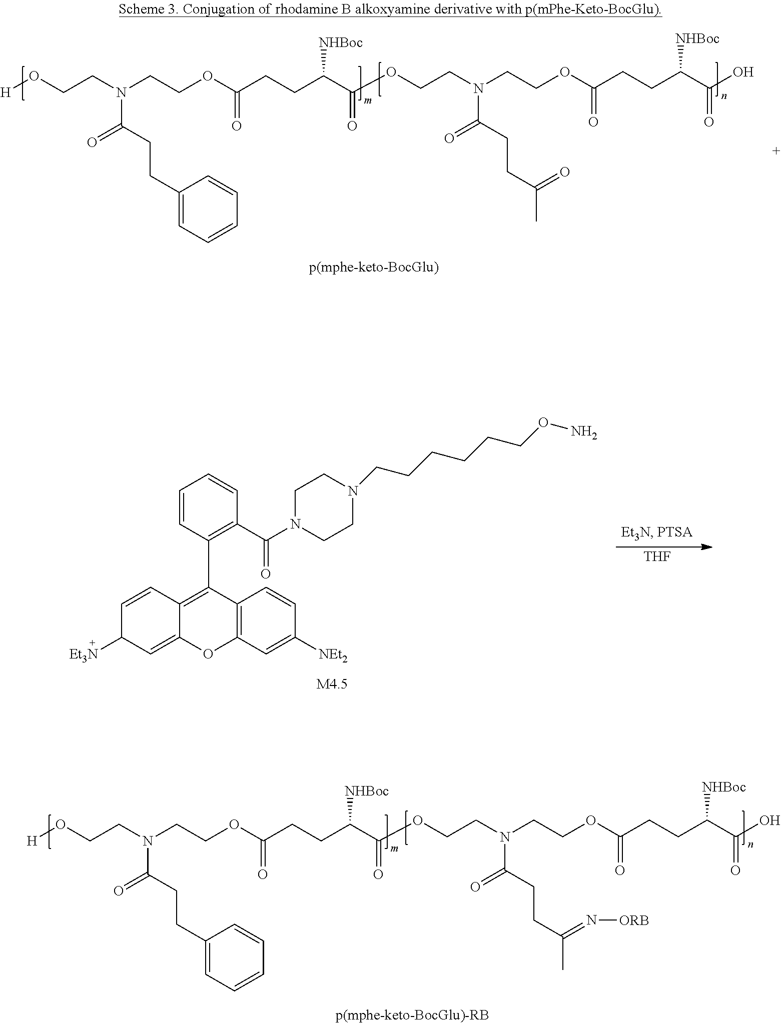

Conjugation of Rhodamine B Alkoxyamine Derivative with p(mPhe-Keto-BocGlu)

1.06 g of p(mPhe-Keto-BocGlu) was dissolved in 6 mL anhydrous THF. 8 mg of M4.5 was dissolved in 4 mL anhydrous THF and 1.5 μL triethylamine was added to M4.5 solution. Then M4.5 solution was added into polymer solution. 4 mg of PTSA was added and the reaction mixture was stirred at RT overnight. The polymer was precipitated three times from MeOH.

Electrospinning of p(mPhe-Keto-BocGlu), 7-(diethylamino)coumarin-3-carboxylic Acid, and Rhodamine B

300 mg p(mPhe-Keto-BocGlu) was dissolved in 5004 chloroform. 75 μL 7-(diethylamino)coumarin-3-carboxylic acid (3 mg) in DMF and 75 μL rhodamine B (1.5 mg) in DMF were added into polymer solution. The mixture was vortexed to mix thoroughly. Then the solution was transferred in to a glass pipette with outlet of 0.5 mm. A thin wire was inserted into the pipette, the end was connected to a direct current. An aluminum foil collecting plate was connected to a grounding electrode. The distance between the tip of glass pipette and aluminum foil was 6 cm. To concentrate the fibers, a copper ring was inserted between the pipette and aluminum foil and the copper ring is 2 cm above pipet tip. 11 kV of direct current was applied to the wire inserted into the pipette and the resulting electrospun fibers were collected on the aluminum foil. The fiber was dried under vacuum overnight. The fiber mat was peeled off from aluminum foil by quick immersion into water and careful removal with tweezers.

Electrospinning of p(mPhe-Keto-BocGlu)-RB and 7-(diethylamino)coumarin-3-carboxylic Acid

p(mPhe-Keto-BocGlu)-RB (300 mg) was dissolved in 5004 chloroform. 150 μL 7-(diethylamino)coumarin-3-carboxylic acid (3 mg) in DMF was added to the polymer solution. The solution was vortexed and transferred to a glass pipette with 0.5 mm outlet. A thin wire was inserted into the pipette, the end was connected to a direct current. An aluminum foil collecting plate was connected to a grounding electrode. The distance between the tip of glass pipette and aluminum foil was 6 cm. To concentrate the fibers, a copper ring was inserted between the pipette and aluminum foil and the copper ring is 2 cm above pipet tip. 12 kV of direct current was applied to the wire inserted into the pipette and the resulting electrospun fibers were collected on the aluminum foil. The fiber was dried under vacuum overnight. Fiber mat was peeled off from aluminum foil by quick immersion into water and careful removal with tweezers.

### UV-Vis Standard Curve of Rhodamine B in PBS

6.36 mg rhodamine B was dissolved in 20 mL PBS. 200 μL solution of this solution was taken and 1.8 mL PBS was added to achieve a 10× dilution. The solution was serially diluted from 100× to 1000×, and the corresponding absorbance of each was measured. Rhodamine B concentration vs absorbance was plotted.

UV-Vis Standard Curve of 7-(diethylamino)coumarin-3-carboxylic Acid in PBS

2.87 mg 7-(diethylamino)coumarin-3-carboxylic acid was dissolved in 15 mL PBS. The above solution was diluted to 30, 40, 50, 60, 70, 80, 90, 100, 150, 200× concentrations. UV absorbance was measured for each solution. 7-(diethylamino)coumarin-3-carboxylic acid concentration vs absorbance was plotted.

### Release Study of Two Dyes

In a 20 mL scintillation vial, 2 cm×2 cm fiber mat of the polyester was immersed in 20 ml PBS. The vial was stored in 37° C. incubator. 2 mL PBS was removed at each time point and replaced with 2 mL fresh PBS, and UV absorbance was measured. These measurements were performed in triplicates. Dye release was calculated according to the UV absorbance standard curve.

### Degradation Study of the Fiber Mat

An electrospun fiber mat (−8 mg) and 20 mL 1×PBS was added to a 20 mL scintillation vial and placed in a 37° C. incubator for a fixed time. Experiments were repeated twice per time point. At each point, PBS was decanted and the sample was rinsed with deionized water. Degradation was analyzed using GPC with DMF as solvent.

### Results

The polymer p(mPhe-Keto-BocGlu) was synthesized from a mixture of diols which contains 80% mPhe monomer, 20% Keto monomer, and Boc protected glutamic acid and polymerized via DIC mediated room temperature polyesterification. Proton NMR of polymer p(mPhe-Keto-BocGlu) with peak designations was determined. The peak at 2.89 corresponds to the methylene group next to the phenyl group with a peak integration of 1.61. The peak at 3.46-3.53 are methylene groups adjacent to the tertiary amine with a peak integration of 4.00. A comparison of the two peaks demonstrates that the polymer contains 80% mPhe pendant group and 20% ketone groups, similar to the feed ratio. This polymer has a Mₙ of 79 kDa, M_{w} of 106 kDa, and a PDI of 1.34. Larger molecular weights are better for electrospinning so this polymer can be electrospun easily. Thermogravimetric analysis shows a decomposition temperature of 219° C., and DSC analysis shoews a glass transition temperature (T_{g}) of 50.1° C. A T_{g} above body temperature is important for integrity of electospun fibers during application. The conjugation of rhodamine B derivative to the polymer was efficiently carried out by oxime bond from the reaction between alkoxyamine and ketone.

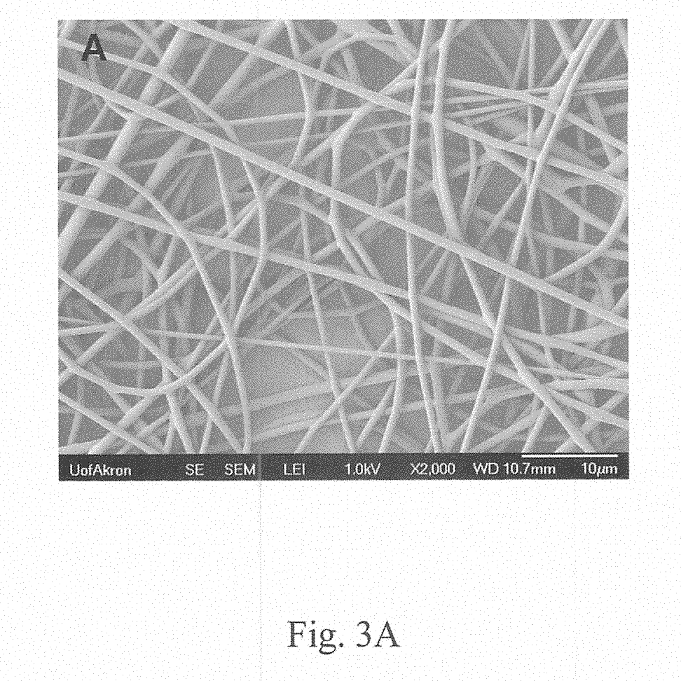

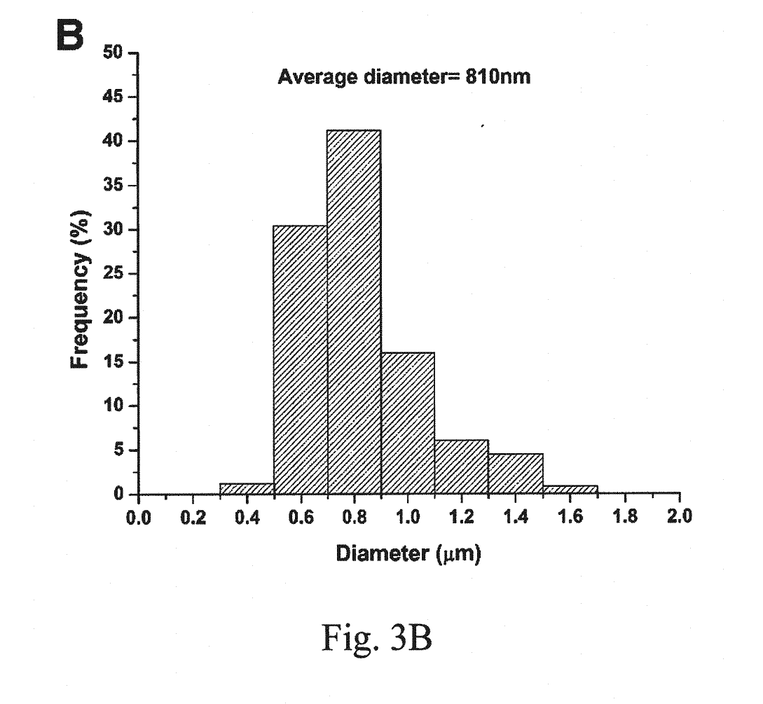

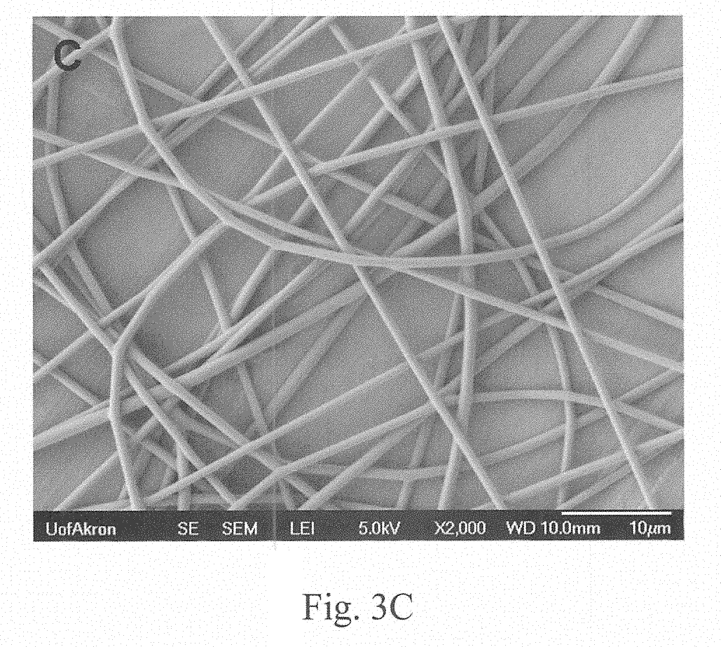

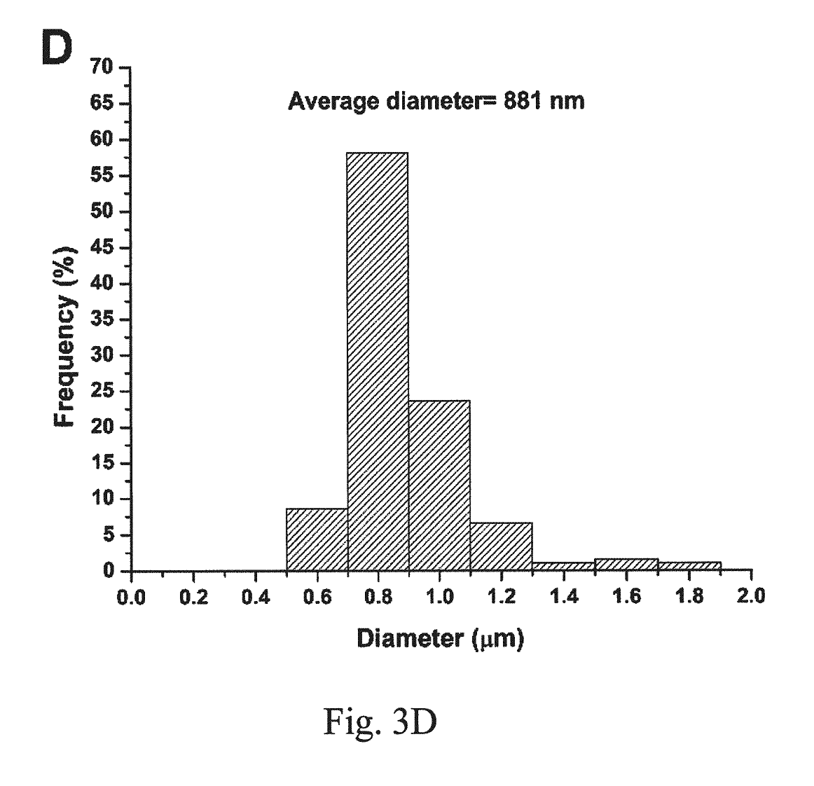

Fiber mats with uniform diameter were obtained by electrospinning mixture of p(mPhe-Keto-BocGlu) and rhodamine B and 7-(diethylamino)coumarin-3-carboxylic acid, or rhodamine B conjugate p(mPhe-Keto-BocGlu)-RB and 7-(diethylamino)coumarin-3-carboxylic acid. FIG. 3A shows the scanning electron microscope (SEM) image of obtained fiber mat from p(mPhe-Keto-BocGlu) mixed with rhodamine B and 7-(diethylamino)coumarin-3-carboxylic acid. The SEM describes continuous fibers with no bead formation. FIG. 3B describes the fiber distribution, analyzed through Nano Measurer 1.2 software. Five SEM images were used for calculation and 50 measurement of diameter for each image was taken. The average nanofiber diameter is 810 nm with a standard deviation of 220 nm. FIG. 3C shows an SEM image of fiber mats from p(mPhe-Keto-BocGlu)-RB mixed with 7-(diethylamino)coumarin-3-carboxylic acid. FIG. 3D describes an average nanofiber diameter of 881 nm with a standard deviation of 180 nm.

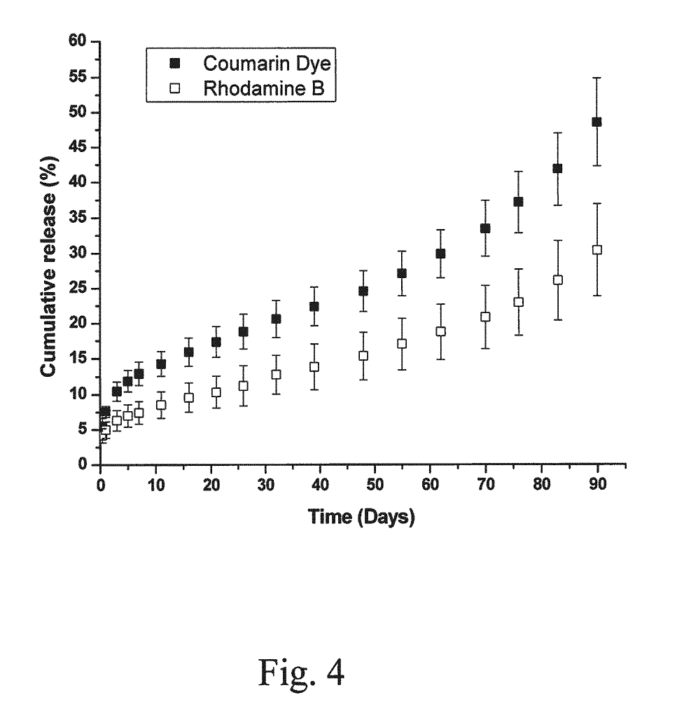

The nanofiber mats were characterized with fluorescence microscopy. Excitation of the nanofiber mat at 345 nm showed a blue fluorescence on the entire mat, which results from coumarin dye fluorescence. Excitation of the nanofiber mat at 555 nm results in yellow fluorescence of the entire mat, describing conjugated rhodamine B dye fluorescence. This means that rhodamine B and coumarin dyes are dispersed in the fiber. FIG. 4 describes cumulative release of coumarin dye and rhodamine B from the fiber mat, made by mixing the two dyes with p(phe-keto-BocGlu). The release was tested up to 90 days. As shown in this figure, the release of each dye has three phases. The release kinetics were characterized by an initial burst release of coumarin from days 0-7, followed by a slower release rate and then by an increase kinetics after day 55.

Rhodamine B release demonstrated similar kinetics. Coumarin dye and rhodamine B were released 12.9±1.6% and 7.4±1.6% respectively from days 0-7 and coumarin dye and rhodamine B were released 27.1±3.2% and 17.0±3.2% respectively after day 55. Coumarin dye and rhodamine B were released at 48.6±6.3% and 30.4±6.5% respectively after day 90. PLA or PLGA fiber mats has been reported to demonstrate encapsulated drug burst release and the released time usually within month.¹⁵⁶⁻¹⁵⁸ The release rate of rhodamine B was lower than coumarin dye, which may indicate stronger polymer-dye interactions between rodamine B compared to coumarin.

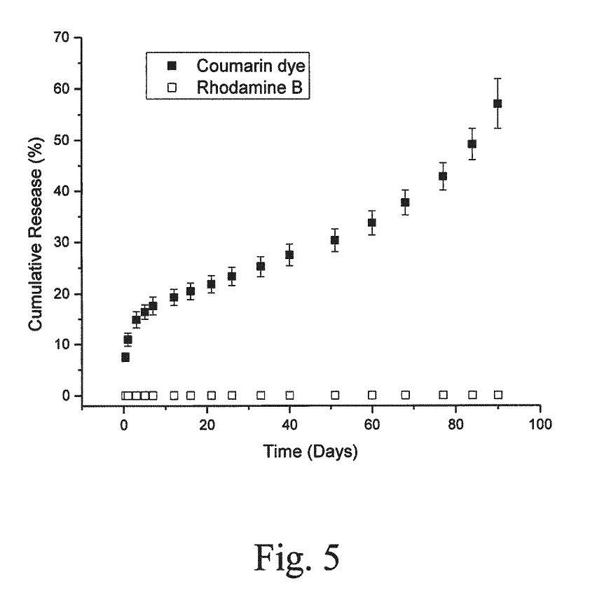

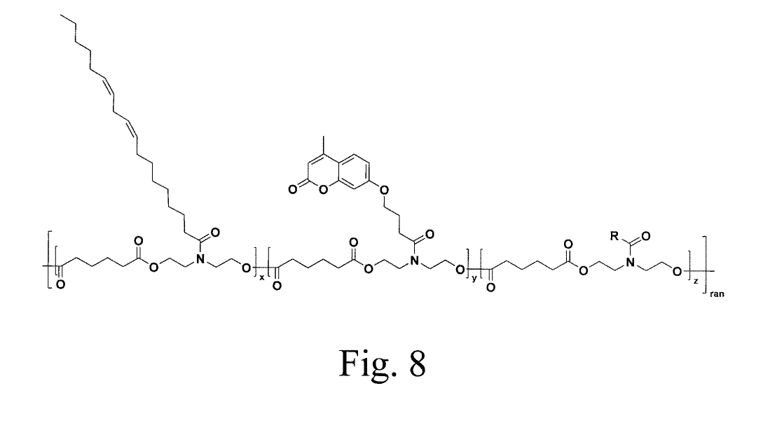

The rhodamine B conjugated polymer with encapsulated coumarin dye p(mPhe-Keto-BocGlu)-RB was also elctrospun to create a fiber mat. FIG. 5 describes cumulative release of coumarin dye and rhodamine B. Coumarin dye encapsulated by RB-conjugated polymer has a similar release profile coumarin dye encapsulated by p(mPhe-Keto-BocGlu), described by FIG. 8 The covalently bonded rhodamine B through oxime bond did not release after 90 days, due to the stable oxime bond in 1×PBS (pH=7.4). Raines and coworkers⁴ studied the hydrolytic stability of hydrazones and oximes and found that oximes are more stable than simple hydrazones. Therefore, oxime bond can be used to tether a bioactive compound.

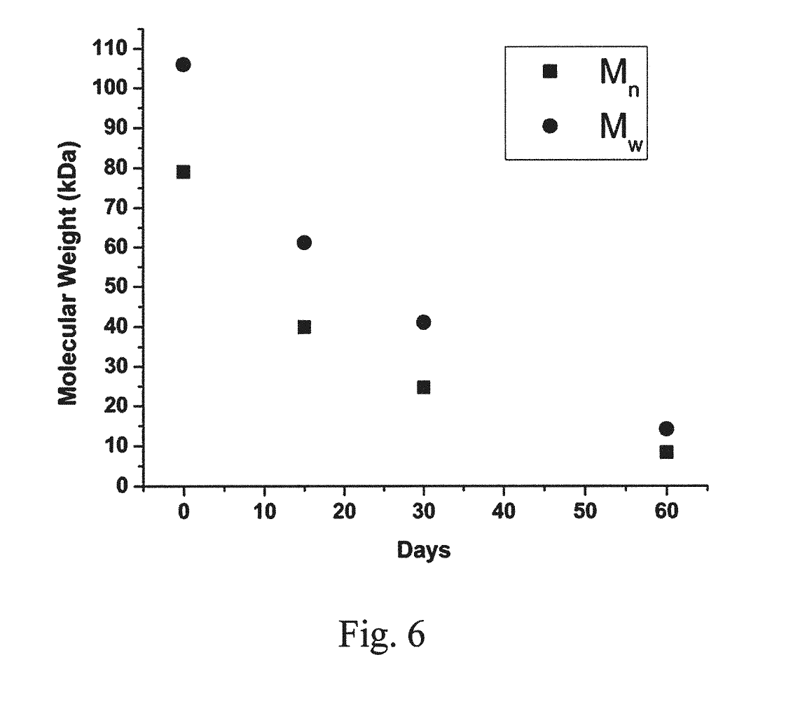

The hydrolytic degradation of p(mPhe-Keto-BocGlu)-RB fiber mat was studied by measuring molecular weights of the fibers at 15, 30, and 60 days. The original and degraded molecular weights are listed in Table 4.1. After 15 days, Mₙ decreased from 79 kDa to 40 kDa, while the M_{w} decreased from 106 kDa to 61 kDa and the PDI increased from 1.34 to 1.53. After 60 days, Mₙ was 25 kDa and M_{w} was 41 kDa, PDI was 1.64. After 60 days, Mₙ was 8.3 kDa and M_{w} was 14.2 kDa. FIG. 6 describes molecular weight vs time immersed in 1×PBS at 37° C. GPC shows traces of degraded polymers.

A polyester with high molecular weight was synthesized from carbodiimide mediated step growth polycondensation of mPhe, ketone bearing monomer and Boc protected glutamicacid. The ketone pendant group was further modified with akoxylamine through oxime bond. The polyester can be electrospun into fiber mats with nanofiber diameter around 800 nm. Coumarin dye and rhodamine B were physically incorporated to fiber by mixing the two dyes with polymer solution. We demonstrated sustained release of both dyes over 90 days. Additionally, a rhodamine B conjugated polymer was synthesized and electrospun with coumarin dye. The release data demonstrates that while coumarin can be released from the fiber mat, the oxime bond between polymer and rhodamine B was stable in PBS and rhodamine B was not released. The fiber degraded over 60 days and showed a decrease in M_{w}, from 106 kDa to 14.2 kDa.

TABLE 1
Molecular weights of p(mPhe-Keto-BocGlu)-RB fiber mats immersed
in 1x PBS at 37° C. for different time.

| Time (Day) | Mn (kDa) | Mw (kDa) | PDI |
|---|---|---|---|
| 0 | 79 | 106 | 1.34 |
| 15 | 40 | 61 | 1.53 |
| 30 | 25 | 41 | 1.64 |
| 60 | 8.3 | 14.2 | 1.53 |

In another experiment, a gluco-cortiscosoid drug, Dexamethasone, used to promote osteogenic growth and as an anti-inflammatory is released as an active agent from the matrix of a functionalized amide polymer. The drug and the amide functionalized polymer are dissolved in a suitable solvent at very dilute concentrations. The concentrations are chosen such that the drug and the polymer are molecularly dispersed. The drug and polymer solutions are mixed and the solvent is rapidly removed by using a rotary evaporator. The polymer drug mixture is then freeze dried in a lyophilizer. This drying combination has been shown to be effective in achieving amorphous drug dispersion in a polymer matrix.

A controlled release matrix is prepared from the amide functionalized polymer with Dexamethasone by additive manufacturing, also known as 3D printing. The polymer drug matrix with a defined geometry is immersed in a physiological buffer solution at 37° C. The solution is changed regularly to not allow the drug to reach its saturation limit in the buffer. The solution is analyzed with a UV-Vis spectrophotometer and the concentration of the released drug is recorded. The graph with the cumulative amount of drug released over eight weeks is shown in figure.

What is claimed is:

1. A controlled release matrix comprising:
a polymer that has backbone selected from polyesters and polyurethanes, and an amide group with a pendant functional group, where the nitrogen atom of the amide group is part of the polymer backbone, and an active compound dispersed in the polymer.

2. The controlled release matrix of claim 1, where the weight percent of the active compound of the total weight of the active compound and polymer is from about 0.1% to about 50%.

3. The controlled release matrix of claim 1, where the functionalized amide polymer includes a unit defined by the formula: 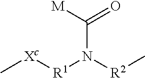
where each X^{c} is an ester group; R¹ and R² may be the same or different and are each hydrocarbon groups; and M is a pendant functional group.

4. The controlled release matrix of claim 1, where the functionalized amide polymer includes a unit defined by the formula: 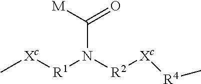
where each X^{c} is a urethane group or an ester group; R¹ and R² may be the same or different and are each hydrocarbon groups; R⁴ is a hydrocarbon group, and M is a pendant functional group.

5. The controlled release matrix of claim 1, where functionalized amide polymer is defined by the formula: 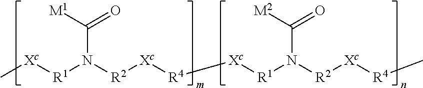
where every X^{c} is a urethane group or every X^{c} is an ester group; R¹ and R² are the same or different and are each hydrocarbon groups; R⁴ is a hydrocarbon group; m and n represent repeating units of the polymer in random or block configuration, and M¹ and M² are different pendant functional groups.

6. The controlled release matrix of claim 1, where functionalized amide polymer is defined by the formula: 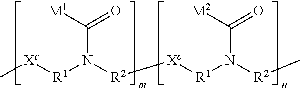
where every X^{c} is a urethane group or every X^{c} is an ester group; R¹ and R² are the same or different and are each hydrocarbon groups; m and n represent repeating units of the polymer in random or block configuration, and M¹ and M² are different pendant functional groups.

7. The controlled release matrix of claim 1, where active agent is selected from small molecules, oligomers, and macromolecules.

8. The controlled release matrix of claim 1, where active agent is selected from active pharmaceutical ingredients, fluorescent dyes, and imaging agents.

9. The controlled release matrix of claim 1, where active agent is selected from peptides, oligonuecleotides, and oligosaccharides.

10. The controlled release matrix of claim 1, where active agent is selected from polysaccharides, proteins, and synthetic polymers.

11. The controlled release matrix of claim 1, where functionalized amide polymer is electrospun.

12. A method of administering an active agent comprising:
supplying a functionalized amide polymer with an active compound dispersed therein to a site in need of the active agent,
where the functionalized amide polymer has backbone selected from polyesters and polyurethanes, and an amide group with a pendant functional group, and the nitrogen atom of the amide group is part of the polymer backbone.

13. The method of claim 12, where the weight percent of the active compound of the total weight of the active compound and polymer is from about 0.1% to about 50%.

14. The method of claim 12, where the functionalized amide polymer includes a unit defined by the formula: 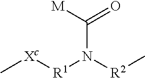
where each X^{c} is an ester group; R¹ and R² may be the same or different and are each hydrocarbon groups; and M is a pendant functional group.

15. The method of claim 12, where the functionalized amide polymer includes a unit defined by the formula: 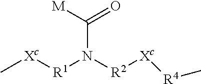
where each X^{c} is a urethane group or an ester group; R¹ and R² may be the same or different and are each hydrocarbon groups; R⁴ is a hydrocarbon group, and M is a pendant functional group.

16. The method of claim 12, where the site in need of the active agent is a bone.

17. The method of claim 12, where the functionalized amide polymer with an active compound dispersed therein is a medical implant.

18. The method of claim 12, where the glass transition temperature of the functionalized amide polymer is greater than 37° C.

19. The method of claim 12, where the glass transition temperature of the functionalized amide polymer is less than 22° C.

20. The method of claim 12, where the glass transition temperature of the functionalized amide polymer is from about 22° C. to about 37° C.

21. The method of claim 12, where the functionalized amide polymer is in the form of a nanoparticles, microparticles, micelles, or vesicle.
